(19) United States
(12) Patent Application Publication
(10) Pub. No.: US 2010/0256439 A1
(43) Pub. Date: Oct. 7, 2010

(54) GANTRY AND SWITCHES FOR POSITION-BASED TRIGGERING OF TMS PULSES IN MOVING COILS

(76) Inventors: M. Bret Schneider, Portola Valley, CA (US); David J. Mishelevich, Playa del Rey, CA (US)

Correspondence Address: SHAY GLENN LLP, 2755 CAMPUS DRIVE, SUITE 210, SAN MATEO, CA, 94403, US

(21) Appl. No.: 12/671,260

(22) PCT Filed: Aug. 12, 2008
(86) PCT No.: PCT/US08/72930

§ 371 Date: Jun. 17, 2010

Related U.S. Application Data
(60) Provisional application No. 60/955,612, filed on Aug. 13, 2007.

(51) Int. Cl. A61N 2/02

(52) U.S. Cl. 600/13

(57) ABSTRACT

When a mechanical frame or gantry is used to move one or more electromagnets about a subject, the pulsed magnetic fields of the magnets need to be triggered, but only when the coil is in an appropriate physical position. Trigger points are established along the movement pathway (e.g., along the support frame) for the electromagnets that trigger the pulsation of the current being supplied to the given electromagnet. Use of the present invention allows firing of a magnetic coil to coordinate with the position of that coil, without need for expensive robotics or computerized motion control.

### CROSS REFERENCE TO RELATED APPLICATIONS

This application claims priority to U.S. Provisional Patent Application Ser. No. 60/954,018, filed on Aug. 13, 2007, titled “GANTRY AND SWITCHES FOR POSITION-BASED TRIGGERING OF TMS PULSES IN MOVING COILS.”

### INCORPORATION BY REFERENCE

All publications and patent applications mentioned in this specification are herein incorporated by reference in their entirety to the same extent as if each individual publication or patent application was specifically and individually indicated incorporated by reference.

### FIELD OF THE INVENTION

The devices and methods described herein relate generally to the triggering of electromagnets used for Transcranial Magnetic Stimulation.

### BACKGROUND OF THE INVENTION

Magnetic stimulation of the body, for example repetitive transcranial magnetic stimulation (rTMS), is most efficiently accomplished if magnetic pulses are discharged from the coil while the coil is in the proper position. While it is possible to simply deliver a constant stream of pulses throughout a stereotyped movement of the coil(s), such an approach is likely to fall short on therapeutic effects and measure high on adverse effects. Properly positioned TMS coils ensure that maximal therapeutic effect is delivered, while minimal adverse effects are elicited. Treatments that make use of properly positioned TMS coils include those methods previously described and disclosed by the inventors in U.S. patent application Ser. No. 10/821,807 “Robotic Apparatus of Stereotactic Transcranial Magnetic Stimulation”.

One means for delivering pulses with a coil in the proper position is to simply deliver a constant stream of pulses, with the assumption that at least some of the time, the coil(s) will be appropriately positioned to induce the desired effects. A disadvantage of this approach is that pulses will likely be also delivered at inappropriate locations, producing unwanted side effects. Consequently, means have been developed by which it can be assured that the coil is pulsed while in the proper physical position. One means for delivering TMS pulses while the coil is in a pre-designated position is a robotic node-based approach, in which a computer instructs a robot regarding the precise position into which an electromagnetic coil is to be moved. Once that position has been achieved, the robot signals the computer that it is now in that position. Only at this point, the computer executes a software function, instructing the TMS device to fire one or more pulses. This method is used by Fox et al. in U.S. Pat. No. 7,087,008.

Using robotics and computerized motion control is both slow and expensive. It would be desirable to have synchronizing means that did not depend on expensive and/or slow computerized robotic control. It would be desirable to adapt a low-cost, reliable, and high-speed gantry system to enable firing of a magnetic coil when at specific physical positions.

### SUMMARY OF THE INVENTION

The present invention involves an approach to synchronizing pulse firing at optimized positions that does not depend upon the use of a computer. This method involves moving the coil(s) in a stereotyped pattern, for example on a motorized gantry, and tripping firing signal switches as the coil moves into a series of firing positions.

In an alternative approach, a mechanical proxy for the coil, for example a timing chain, coordinates timing of firing relative to coil positioning.

In yet another alternative embodiment, timing between firing and coil positioning is coordinated by the timecode encoding of both the movement of the coil, for example on a gantry, and the triggering of the pulses. With the timing of pulses synchronized to the time code of the gantry, firing will occur only when the coil is in the proper position, provided that all system components operate in a manner that is true to their time base. This approach may be accomplished by electronic means, using a common clock that is attached to both gantry and pulse generation units (or by using multiple synchronized clocks).

### BRIEF DESCRIPTION OF THE DRAWINGS

FIG. 1 shows an embodiment in which one or more coils orbiting a circular gantry are triggered to fire by a post located at each predetermined station.

FIG. 2 illustrates the use of optical switches at two stations on a gantry.

FIG. 3A shows a coil array that moves back and forth along a semicircular arc, while position of the array is indicated at a point on the gantry remote from the actual coil location.

FIG. 3B shows further details of the embodiment outlined in FIG. 3A

### DETAILED DESCRIPTION OF THE INVENTION

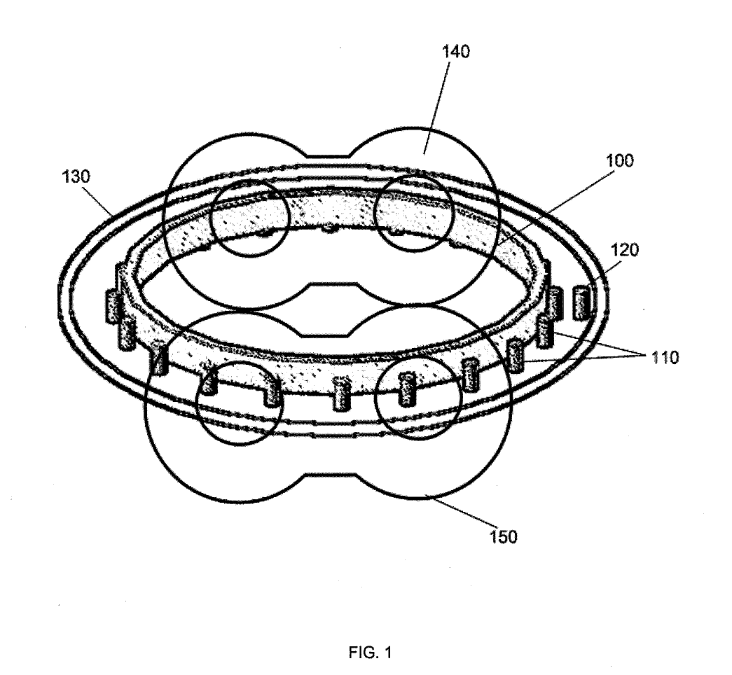

FIG. 1 illustrates an embodiment involving a circular frame gantry 100. Positioned around the perimeter of frame 100 are trigger points 110. These trigger points need not be uniformly distributed around the frame perimeter. Embodiments of trigger the devices 120 to be triggered when trigger points 110 are in proximity as shown in FIG. 1 may include an electromechanical switch (such as a standard normally-open push-button switch (Jameco Electronics, Belmont, Calif.)) or switches held on a support 130 tripped by physical or non-physical contact with trigger points. Alternative embodiments for the switches may include Hall effect sensors, reed switches, interruption of light beams, interruption of audio beams, microphones where the trigger points emit audio, or radio-frequency devices such as RFID tags, or similar devices. For locations that should not be stimulated (when it is desired to protect underlying tissue), the trigger is not installed or otherwise not enabled such that no magnetic pulse firing at that trajectory will occur. The operator may place the trigger positions manually. The locations may be determined by finding the appropriate positions on a map related to target locations or be calculated using a computer during a pre-procedure treatment planning process. For example, triggers may be positioned or set based on calculated beam trajectories produced by radiosurgery treatment planning software such as MultiPlan® Treatment Planning System (Accuray Inc., Sunnyvale, Calif.). In some variations the triggers are positioned based on treatment plans derived or created as part of a pre-treatment step for the patient. This treatment plan may include one or more maps of the patient's anatomical (e.g., brain) structures, e.g., using one or more imaging modalities. Configuration of trigger points so as to make them active or inactive when the coil passes by may be conducted during the pre-procedure process by loading the treatment plan into a configuration utility. For example, trigger points that are required to be active in order to deliver energy to a target in accordance with the treatment plan, and which are not to be avoided as per the treatment plan, are configured in the “active” position. In the example shown in FIG. 1, coils 140 and 150 travel on circular frame gantry 100. Any appropriate track or gantry may be used.

Treatment plans for medical energy delivery systems, including stereotactic radiosurgery, radiotherapy and ultrasound are well known in the art. In general these systems include means for calculated the predicted dose to be delivered to a specified target, while avoiding, or limiting dose to specified structures. Examples include the MultiPlan software by Accuray, Inc., Santa Clara, Calif.

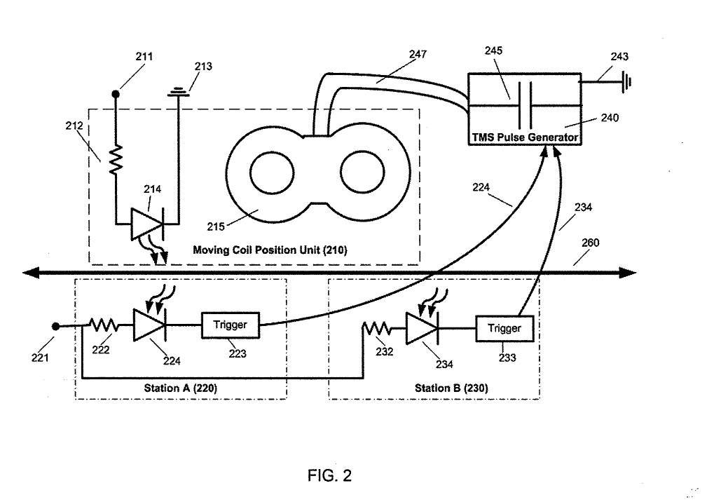

FIG. 2 illustrates the use of optical switches at two stations on a gantry. Moving Coil Position Unit 210 is composed principally of TMS coil 215 and light-emitting diode (LED) 214, and is moved along a stereotyped path 260 along a gantry (not shown). LED 214 draws power from voltage supply 211, as limited by resister 212, and grounded by ground 213. Trajectory line 260 shows a portion of the stereotyped path that the coil moves with respect to the gantry (represented by the area below trajectory line 260). Within the gantry below trajectory line 260, two stations—Station A 220 and Station B 230 are located at different physical locations on the gantry. Both Station A 220 and Station B 230 are optical detection switches. For example, in Station A 220, photodiode 224 receives power from voltage supply 221, as limited by resister 222. When Moving Coil Position Unit 210 moves into place on the gantry next to Station A 220, light from LED 214 strikes photodiode 224, dropping its resistance and allowing current to flow through to trigger 223, which transmits a trigger signal via line 224 in order to signal the TMS pulse generator unit 240 to discharge its capacitors 245. The electrical pulse released from capacitors 245 is sent down cable 247 to TMS coil 215. As the automated movement of the Moving Coil Position Unit 120 moves away from Station A 220, light will no longer reach photodetector 223. Until an appropriate station with the requisite detector is reached, no further triggers will be sent to TMS Pulse Generator 240. Subsequently, When Moving Coil Position Unit 210 moves into place on the gantry next to Station B 230, light from LED 214 strikes photodiode 234, dropping its resistance and allowing current to flow through to trigger 233, which transmits a trigger signal via line 234 in order to signal the TMS pulse generator unit 240 to discharge its capacitors 245. The electrical pulse released from capacitors 245 is sent down cable 247 to TMS coil 215. During the pre-procedure time, automated configuration by the treatment planning system may be accomplished. During this process, specific optical switch positions are designated as “on” or “off” depending the specific target and structures to be avoided in the present treatment plan. An alternative embodiment is to have a single receiver (e.g., light sensor) and multiple transmitters (e.g., light emitters).

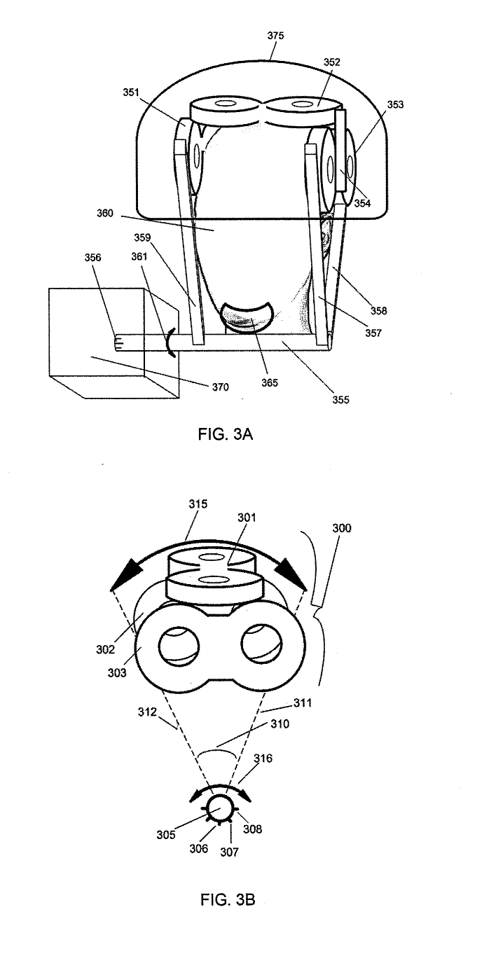

FIG. 3A shows coil array 300, which includes coil 301, coil 302 and coil 303. In this particular example, each component coil is a double air-core coil. Coil array 300 is able to move as an integral whole, back and forth along a path described by arc 315 and angle of travel 310, the lateral bounds of which are described by lines 311 and 312. This semicircular path is designed to accommodate the curvature of the human skull while moving from a dorsal anterior position to a dorsal posterior position. The coil array is arranged in a semicircular arc, while the position of the array is indicated at a point on the gantry that is remote from the actual coil location. Coil array 300 is rigidly affixed to a gantry (not shown in 3A, but represented as gantry struts 357 and gantry tiller 355 in FIG. 3B), which lies substantially along the plane of line 311 and 312. This gantry is moved back and forth by gantry tiller 305, which is endowed firing switch markers 306, 307 and 308. These may be, for example, physical features such as protuberances or recesses, or may be optical markers such as line patterns, or optically readable symbols for an optical encoder.

An alternative embodiment is to move the coil back and forth, rotating in a horizontal pane with the axis of rotation in the center of the skull.

FIG. 3B shows further details of the embodiment outlined in FIG. 3A. A patient 360 is placed between gantry structures including a gantry bar 357, gantry bar 358 and gantry bar 359 (the companion gantry bar to 359 (equivalent to gantry bar 358 relative to gantry bar 357) is not shown), resting his or her chin on chin rest 365. A coil array including coil 351, coil 352 and coil 352 are held in a configuration and stabilized by means including connector bar 354. The array is affixed to gantry bars 357 and 358, and gantry tiller 355, preferably using moveable connections, for individualized size and targeting adjustments. Gantry tiller includes firing switch markers 356. These may be physical features such as protuberances or recesses, or may be optical markers such as line patterns. Gantry tiller 355 is turned back and forth along arc 361 by motor unit 370, which may be, for example, a servo or step motor. In this manner, coils 351, 352 and 353 are moved in an arc over the head of patient 360. coil array and gantry may be partially or completely covered by enclosure 375, for enhancement of safety and aesthetic appeal. Enclosure 375 can be air cooled to dissipate heat generated by the coil array.

As with previous embodiments discussed, prior to use with a specific patient, the operator may place the trigger positions manually. The locations can be determined by finding the appropriate positions on a map related to target locations or be calculated using a computer. For example, a method o treatment may include a pretreatment phase in which a map of the patient's anatomy is used to help place one or more triggers. The treatment map may include the calculation of the energy to be applied to one or more regions. Further, pre-treatment may include the step of determining the position of one or more triggers to activate stimulation. Finally, the timing or speed of the motion of the treatment device (e.g., the magnet(s) along the gantry) may be determined. The pre-treatment steps may include setting up the device and preparing the patient based on the pre-treatment determinations (the treatment map). After pre-treatment is completed, the patient may be positioned in the device (if they have not already been positioned) and the treatment step may begin, moving the magnet(s) on the gantry, and triggering the application of energy based on the pre-positioned triggers.

In an alternative embodiment, a given trigger position may be automatically enabled by during an electronic configuration process involving input of a completed treatment plan. Because the treatment plan calls for specific pulse trajectories, the closest matching coil positions may be automatically enabled. This may be accomplished by any appropriate method, including using a computer system to differentially register or ignore specific switch output positions in accordance with the configuration settings.

As noted previously, a variety of types of trigger device may be used and the invention is not limited by the particular variations specifically discussed herein.

### REFERENCES

Traad, Monique. “A Quantitative Positioning Device For Transcranial Magnetic Stimulation”. Engineering In Medicine and biology Society, 1990. Proceedings of the Twelfth Annual International Conference of the IEEE. Philadelphia, Pa., Nov. 1-4, 1990. p. 2246.

Fox et al., Apparatus and methods for delivery of transcranial magnetic stimulation, U.S. Pat. No. 7,087,008.

Walsh V, and A. Pascual-Leone, “Transcranial Magnetic Stimulation: A Neurochronometrics of Mind,” MIT Press, Cambridge, Mass. 2003.

U.S. patent application Ser. No. 10/821,807 “Robotic Apparatus of Stereotactic Transcranial Magnetic Stimulation”. Schneider M B and Mishelevich D J.

What is claimed is:

1. A system for delivering magnetic energy to specified sub-surface brain structures comprising:
a motorized gantry having a predetermined pathway;
one or more electromagnetic coils configured to travel on the gantry in a route defined by predetermined pathway of gantry; and
one or more switches at predetermined locations along the pathway of the gantry; wherein the switches are configured to trigger one or more electromagnetic pulses as electromagnetic coil reaches each switch.

2. The system of claim 1, wherein the switches are electromechanical.

3. The system of claim 1, wherein the switches are electro-optical.

4. A system for delivering magnetic energy to specified sub-surface brain structures comprising:
a motorized, moving gantry having a predetermined pathway;
one or more electromagnetic coils configured to travel the gantry pathway; and
one or more position markers on the gantry along the pathway from which the position of the electromagnetic coils can be inferred; wherein the system is configured to trigger one or more electromagnetic pulses from the electromagnetic coils based on the position markers.

5. A treatment planning and device configuration system comprising:
one or more electromagnetic coils that are configured to move on a motorized gantry in a predetermined pathway; and
one or more switches responsive to the position of said electromagnetic coils on the gantry; wherein the switches are configured to be enabled or disabled in accordance with the trajectories required by a treatment plan.

6. A method of applying transcranial magnetic stimulation, the method comprising:
performing a pre-treatment phase including determining a treatment plan for triggering transcranial magnetic stimulation at one or more desired locations around the patient;
setting a plurality of trigger points along a movement pathway of a gantry based on the treatment plan, wherein the gantry is configured to support the movement of one or more electromagnetic coils along the movement pathway; and
moving the one or more electromagnetic coil along the movement pathway and triggering transcranial magnetic stimulation when the one or more electromagnetic coils reach a trigger point.
